SEQUENCE LISTING (8 sequences)

SEQ ID NO: 1 - protein, 26 aa, Homo sapiens
MDPQTAPSRALLLLLFLHLAFLGGRS

SEQ ID NO: 2 - protein, 76 aa, Homo sapiens
HPLGSPGSASDLETSGLQEQRNHLQGKLSELQVEQTSLEPLQESPRPTGVWKSREVATEGIRGHRKMVLYTLRAPR

SEQ ID NO: 3 - protein, 32 aa, Homo sapiens
SPKMVQGSGCFGRKMDRISSSSGLGCKVLRRH

SEQ ID NO: 4 - protein, 153 aa, Homo sapiens [sequence omitted]

SEQ ID NO: 5 - protein, 126 aa, Homo sapiens
MHLSQLLACALLLTLLSLRPSEAKPGAPPKVPRTPPAEELAEPQAAGGGQKKGDKAPGGGGANLKGDRSRLLRDLRVDTKSRAAWARLLQEHPNARKYKGANKKGLSKGCFGLKLDRIGSMSGLGC

SEQ ID NO: 6 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 7 - protein, 227 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSVSHPYSQDLEGKGEWGP

SEQ ID NO: 8 - protein, 13 aa, Homo sapiens
PQTAPSRALLLLL

(19) United States
(12) Patent Application Publication
(10) Pub. No.: US 2005/0244904 A1
(43) Pub. Date: Nov. 3, 2005

(54) Diagnostics based on signal peptide detection

(76) Inventor: Leong Ng, Leicester (GB)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/977,334

(22) Filed: Oct. 29, 2004

Related U.S. Application Data
(60) Provisional application No. 60/542,647, filed on Feb. 6, 2004.

(30) Foreign Application Priority Data
Oct. 29, 2003 (GB) 0325279.8

(51) Int. Cl. G01N 33/53; G01N 33/537; G01N 33/543

(52) U.S. Cl. 435/7.92

(57) ABSTRACT

The invention provides methods and a kit for detecting peptides and proteins, which may be indicative of heart disease by detecting signal peptide levels for a protein in a subject's bodily fluid.

### CROSS REFERENCE TO RELATED APPLICATIONS

This application claims the benefit of priority to U.S. Provisional Patent Application Ser. No. 60/542,647, filed on Feb. 6, 2004, and United Kingdom Application No. 0325279.8, filed on Oct. 29, 2003, which applications are hereby incorporated by reference in their entirety.

### BACKGROUND OF THE INVENTION

Signal peptide sequences are found in many eukaryotic proteins that are synthesized within cells, and are thought to direct proteins towards the secretory pathway.

Proteins containing signal peptide sequences are targeted towards the endoplasmic reticulum and are attached to the endoplasmic reticulum membrane through a hydrophobic section of a signal peptide (Martoglio & Dobberstein, Trends Cell Biol 1998; 8: 410-5). Signal peptide sequences have a characteristic structure which includes a short positively-charged N-terminal region (n-region), a middle hydrophobic section (h-region), and a neutral polar C-terminal section where the signal sequence is cleaved from the protein to be secreted (Nielsen et al, Protein Eng 1997; 10: 1-6).

During the process of secretion, a signal peptide, which attaches a protein to the endoplasmic reticulum membrane, is cleaved by specific enzymes such as signal peptidases, and the protein is released into the endoplasmic reticulum lumen (Paetzel et al, Nature 1998; 396: 186-90; Weihofen et al, Science 2002; 296: 2215-8). The remaining signal peptide remnant is thought to remain anchored to the membrane due to its hydrophobic mid-section. Although some signal peptide sequences may be released into the cell cytoplasm by protease activity, where they may be further degraded, signal peptides are not expected to circulate in plasma or other bodily fluids.

Recent evidence suggests that there may be some proteases that could cleave the signal peptide within its hydrophobic trans-membrane region (Weihofen et al, J Biol Chem 2000; 275: 30951-6; Lyko et al, J Biol Chem 1995; 270: 19873-8), so that the remaining N-terminal fragment could be released into the cytosol and interact with other proteins such as calmodulin (Martoglio et al, EMBO J. 1997; 16: 6636-45). Changes in composition of the signal sequence could also alter the amount of protein that is secreted (Kallio et al, J Clin Endocrinol Metab 2001; 86: 5348-52).

Secreted proteins are frequently used as markers of disease given their relative ease of detection in plasma and other bodily fluids. However, not all secreted proteins are indicative of early stages of disease. Accordingly, new markers are needed for the effective, early detection of numerous disease states.

### SUMMARY OF THE INVENTION

The disclosed invention is based on the unexpected finding that signal peptide sequences are secreted into plasma or other bodily fluids, and that the levels of a secreted signal peptide are directly proportional to the levels of the cleaved parent protein. Thus, the measurement of a signal peptide in bodily fluid is indicative of the level of that protein.

In one aspect, the invention provides a method for determining the level of a protein by detecting the level of the corresponding signal peptide in a subject's bodily fluid. This determination can be made using, for example, an immunoassay to measure the signal peptide alone or in conjunction with the mature protein from which it was cleaved. These measurements can establish a relationship between the level of a signal peptide and its parent protein, such as extrapolating a bodily fluid level of the parent protein from the measured level of a related signal peptide. Alternatively, where the level of the parent protein is a marker, e.g. predictive or indicative of a disease, the level of a related signal peptide can be used in detection of that disease.

The subject from which bodily fluid is obtained may be a mammalian subject and preferably is a human subject. Bodily fluids include, but are not limited to, plasma, interstitial fluid, urine, whole blood, serum, or saliva.

In a second aspect, the invention provides kits for detecting and/or quantitating the amount of a signal peptide in a subject's bodily fluid at home or in a doctor's office. Such a kit may comprise one or more reagents for measuring the level of a signal peptide in bodily fluid. The one or more reagents may comprise an antibody that is immunospecific for a signal peptide.

Signal peptides may be secreted earlier than their corresponding parent proteins, and thus, may serve as early indicators for disease detection. Other features and advantages will be appreciated based on the following Detailed Description and claims.

### BRIEF DESCRIPTION OF THE DRAWINGS

FIGS. 1A-C show the amino acid sequences of the signal peptide of brain natriuretic peptide (BNP-SP) (SEQ ID NO:1); N-BNP (SEQ ID NO:2); and BNP (SEQ ID NO:3), respectively (NCBI database accession P16860).

FIG. 2 shows the amino acid sequence of human atrial natriuretic peptide (ANP) precursor (SEQ ID NO:4) (NCBI database accession P01160). The first 25 amino acids of the protein (in bold) represent the ANP-signal peptide (amino acids 1-25 of SEQ ID NO:4). Mature ANP is underlined (amino acids 124-151 of SEQ ID NO:4).

FIG. 3 shows the amino acid sequence of human C-type natriuretic peptide (CNP) precursor (SEQ ID NO:5) (NCBI database accession P23582). The first 23 amino acids of the protein (in bold) represent the CNP-signal peptide (amino acids 1-23 of SEQ ID NO:5). Mature CNP is underlined (amino acids 105-126 of SEQ ID NO:5).

FIG. 4 shows the amino acid sequence of C-reactive protein (CRP) precursor (SEQ ID NO:6) (NCBI database accession PO₂₇₄₁). The first 18 amino acids of this protein (in bold) represent the CRP-signal peptide (amino acids 1-18 of SEQ ID NO:6).

FIG. 5 shows the amino acid sequence of prostate specific antigen (PSA) (SEQ ID NO:7) (NCBI database accession CAD54617). The first 17 amino acids of this protein (in bold) represent the PSA-signal peptide (amino acids 1-17 of SEQ ID NO:7).

FIG. 6 is a standard curve for signal peptide of BNP. RLU is the ratio (in relative light units) of chemiluminescence counts relative to that at zero level of the peptide.

FIG. 7 is a box plot of plasma N-BNP levels in Heart Failure and Normal patients.

FIG. 8 is a box plot of plasma BNP-SP levels in Heart Failure and Normal patients.

FIG. 9 is a graph showing the correlation of Plasma BNP-SP and N-BNP levels (▴, normal controls; ∘, heart failure patients).

FIG. 10 shows Receiver Operating Characteristic curves for the diagnosis of heart failure, using N-BNP or BNP-SP (Signal Peptide of BNP) ( [symbol] , N-BNP; [symbol] , BNP-SP).

### DETAILED DESCRIPTION OF THE INVENTION

1. Definitions

For convenience, before further description of the disclosed invention, certain terms employed in the specification, examples, and appended claims are provided here.

The singular forms “a”, “an”, and “the” include plural references unless the context clearly dictates otherwise.

“ANP” refers to atrial natriuretic peptide, the first described peptide in a family of hormones which regulate body fluid homeostasis (Brenner et al., Physiol. Rev. 1990; 70: 665-99).

The term “antibody,” as used herein, refers to binding molecules including immunoglobulin molecules and immunologically active portions of immunoglobulin molecules, i.e., molecules that contain an antigen binding site that specifically bind an antigen. The immunoglobulin molecules useful in the invention can be of any class (e.g., IgG, IgE, IgM, IgD and IgA) or subclass of immunoglobulin molecule. Antibodies include, but are not limited to, polyclonal, monoclonal, bispecific, humanized, and chimeric antibodies, single chain antibodies, Fab, F(ab′), and F(ab′)₂ fragments, fragments produced by a Fab expression library, anti-idiotypic (anti-Id) antibodies, and epitope-binding fragments of any of the above. An antibody, or generally any molecule, “binds specifically” to an antigen (or other molecule) if the antibody binds preferentially to the antigen, and, e.g., has less than about 30%, preferably 20%, 10%, or 1% cross-reactivity with another molecule. Portions of antibodies include Fv and Fv′ portions.

“BNP,” “N-BNP,” or “proBNP” refers to a cardiac derived peptide hormone that circulates in the blood and exerts potent cardiovascular and renal actions. Mature human BNP consists of a 32 amino acid peptide containing a 17 amino acid ring structure formed by two disulfide bonds.

The term “bodily fluid” includes all fluids obtained from a mammalian body, including, for example, whole blood, plasma, urine, interstitial fluid, lymph, gastric juices, bile, serum, saliva, sweat, and spinal and brain fluids. Furthermore, a bodily fluid may be either processed (e.g., serum) or unprocessed.

“CNP” refers to C-type natriuretic peptide (Stingo et al., Am. J. Physiol., 1992; 263: H1318-21).

“Comprise” and “comprising” are used in the inclusive, open sense, meaning that additional elements may be included.

“CRP” refers to C-reactive protein (Blake & Ridler, J Am Coll Cardiol. 2003; 41 (4 Suppl S):37S-42S).

The terms “detection” and “detect,” as used herein, refer to determining the presence or absence of a marker.

The term “diagnosis,” as used herein, refers to the identification of a disease in a subject or the subject's susceptibility to develop the disease.

The term “disease,” as used herein, refers to any illness, disorder, or abnormal condition of body function or structure that is considered to be harmful to the affected individual.

The term “heart disease,” as used herein, refers to a wide range of abnormalities of the heart, coronary vasculature, or blood vessels surrounding the heart including underlying conditions, such as, ischemia (including, for example, atherosclerosis (coronary artery disease), embolism, congenital heart defects, anemia, lung disease, and abnormal stimulation (e.g., sympathomimetic abuse)), hypertension (including, for example, systemic hypertension (e.g., primary and secondary) and pulmonary hypertension (e.g., chronic obstructive pulmonary disease, restrictive lung disease, pulmonary embolism, and morbid obesity)), valvular disease (including, for example, mitral valve disease, aortic valve disease, tricuspid valve disease and pulmonary valve disease), heart muscle disease (including, for example, ischemic cardiomyopathy, dilated cardiomyopathy, hypertensive cardiomyopathy, hypertrophic cardiomyopathy, restrictive cardiomyopathy, and specific heart muscle disease resulting from cardiac infection, toxins, metabolites, neuromuscular disease, storage disorders, infiltration disorders, and immunologic disorders), pericardial disease, rheumatoid heart disease, neoplastic heart disease (including, for example, primary cardiac tumors), and coronary vasospasm (including, for example, drug induced vasospasm) that may manifest as angina (including, for example, stable angina, unstable angina and Prinzmetal's variant angina), myocardial infarction, chronic ischemic heart disease, and sudden cardiac death.

The term “immunoassay,” as used herein, refers to an assay that utilizes an antibody to specifically bind to a protein.

The term “marker” or “marker protein,” as used herein, refers to the amount of polypeptide or peptide (e.g. signal peptide) in bodily fluid that is indicative of a disease and that can be detected and/or quantitated.

As used herein, the term “natriuretic peptide” includes a native ANP, BNP, or CNP, portions of, variants of, or chimeras thereof.

“PSA” refers to prostate specific antigen (So et al., Can J Urol, 2003, 10: 2040.)

The terms “quantify” and “quantitate,” as used herein, refer to the process of measuring the amount of a marker (e.g., in terms of its concentration, mass, moles, or volume in a sample).

A “reagent” refers to a substance or molecule that binds or interacts with a polypeptide or peptide.

A “subject” refers to a human or a non-human animal.

2. General

It is shown for the first time that a signal peptide from human brain natriuretic peptide is present in normal human plasma, and its levels are elevated to the same extent as the levels of N-BNP, a recognized marker of heart disease. Based on this finding, in one aspect, the invention features methods for measuring the signal peptide of BNP (BNP-SP) to diagnose heart disease where the BNP system is up-regulated, resulting in elevated plasma N-BNP or BNP levels. The finding that a signal peptide is secreted with the mature secreted protein (in this case BNP-SP and N-BNP) is unexpected.

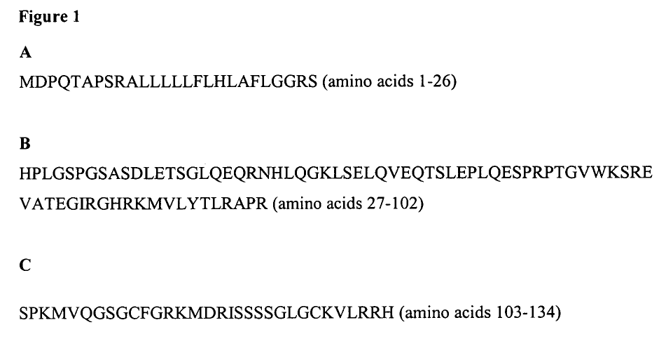

In heart disease, there is evidence of up-regulation of the brain natriuretic peptide system, with increased plasma levels of brain natriuretic peptide (BNP) (Wei et al, Circulation 1993; 88: 1004-9; McDonagh et al, Lancet 1998; 351: 9-13), and N-terminal brain natriuretic peptide (N-BNP) (Hunt et al, Clin Endocrinol 1997; 47: 287-96; Hughes et al, Clinical Science 1999; 96: 373-380). Both the active peptide (BNP) and the inactive peptide (N-BNP) are derived from a single precursor protein (proBNP). The whole protein (FIG. 1) (Sudoh et al, Biochem Biophys Res Commun 1989; 159: 1427-34) consists of a signal peptide sequence (amino acids 1-26) and proBNP (amino acids 27-134) from which N-BNP (amino acids 27-102) and BNP (amino acids 103-134) are derived. The release of proBNP from cardiac myocytes in the left ventricle, and the increased production of BNP is triggered by myocardial stretch, myocardial tension, and myocardial injury.

As described above, signal peptide sequences are believed to direct a protein towards the secretory pathway. After proBNP has been cleaved off from its signal sequence, which anchors proBNP to the endoplasmic reticulum membrane, the BNP signal sequence has no other known function and has never been described as a secreted peptide. Where detection of the BNP signal peptide is made by means of an antibody, the antibody may be raised against any part of the signal peptide, including the part having the sequence PQTAPSRALLLLL (SEQ ID NO:8).

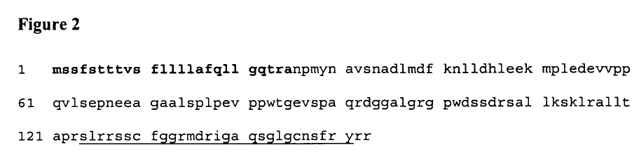

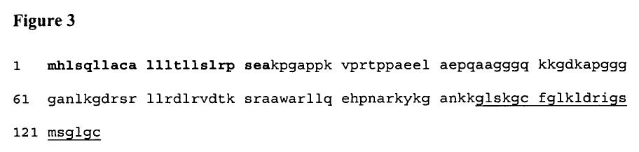

In further embodiments, other signal peptides, e.g. those derived from other natriuretic peptide such as atrial natriuretic peptide (ANP) (FIG. 2) (Hall, Eur J Heart Fail, 2001, 3:395-397) and C-type natriuretic peptide (CNP) (FIG. 3), may also be found in human plasma and reflect the status of the hormonal or protein secretory systems from which they were derived.

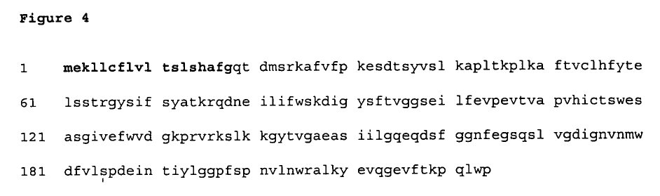

Alternatively, C-reactive protein (CRP) or the CRP-signal peptide may be used to detect heart disease. High sensitivity plasma CRP levels can be used in the detection and diagnosis of ischemic heart disease in apparently healthy people, or in the risk stratification of patients after acute coronary syndromes (Blake & Ridker, J Am Coll Cardiol. 2003; 41(4 Suppl S):37S-42S). FIG. 4 shows the amino acid sequence of CRP and the CRP-signal peptide, which will be cleaved off from the mature protein.

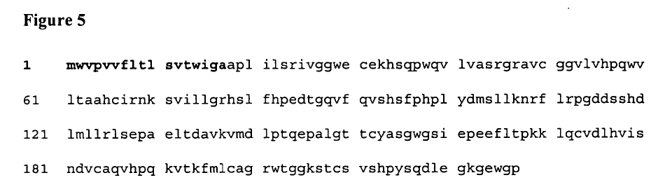

In another embodiment, prostate cancer may be detected by measuring the level of prostate specific antigen (PSA)-signal peptide in bodily fluid. PSA is a marker of prostate cancer and may be used to detect and diagnose prostate cancer. FIG. 5 shows the amino acid sequence of PSA and the PSA-signal peptide, which will be cleaved off from the mature protein.

3. Methods for Detecting a Signal Peptide

A signal peptide sequence can be determined in a variety of ways known to one of skill in the art. For example, signal peptide sequences can be determined using analytical software, such as the software found at the Centre for Biological Sequence Analysis (see the World Wide Web at cbs.dtu.dk/services/SignalP/mailserver). Such signal peptide sequences may then be detected and/or quantitated in bodily fluid.

While signal peptides may comprise unique sequences corresponding to a particular parent protein, signal peptides typically comprise a common structure including a positively charged N-terminal domain (n-region), a central hydrophobic region (h-region), and a neutral, but polar C-terminal region (c-region). The n-region of a signal peptide may be approximately 2 to 15 amino acid residues in length and may comprise one or more arginine or lysine residues. The n-region is typically polar and carries a net positive charge, but is not restricted in amino acid content or length. The central h-region is approximately 6 to 15 amino acid residues in length and predominantly comprises hydrophobic amino acid residues (e.g., leucine, alanine, valine, isoleucine, glycine, phenylalanine, methionine, and tryptophan) and is devoid of strongly polar or charged amino acid residues (e.g., lysine, arginine, histidine, aspartic acid, glutamic acid, and proline). High leucine or alanine content in the h-region may cause signal peptides to adopt an alpha-helical configuration in apolar environments. The c-region is less hydrophobic and typically comprises neutral and polar amino acid residues, but is not limited in amino acid content. This region also contains signals that are recognized by signal peptidases. These signals are located at positions −1 and −3 of the signal peptide and must be small and neutral for cleavage to occur correctly. The amino acids at these positions are typically alanine or glycine with a turn inducing residue at −6 (e.g., glycine or proline) with respect to the cleavage site.

The level of a signal peptide may be detected and/or quantitated in bodily fluid, such as plasma, whole blood, urine, interstitial fluid, lymph, gastric juices, bile, serum, saliva, sweat, and spinal and brain fluids. Furthermore, bodily fluid may be either processed (e.g., serum) or unprocessed. Methods of obtaining bodily fluid from a subject are well-known to one of skill in the art.

In the invention, the level a signal peptide may be detected and/or quantitated in bodily fluid by using an antibody directed against a particular signal peptide. Specific antibodies to a signal peptide may be directed against an entire signal peptide or any portion of a signal peptide. Since signal peptides do not share a common amino acid sequence, it is possible to generate specific antibodies against a specific signal peptide. Bioinformatic approaches, well-known to one of skill in the art, may be used to determine homology between a signal peptide and other known sequences. Bioinformatic approaches may also be used to determine unique sequences within a signal peptide.

Antigenic fragments within a signal peptide may be identified by methods well-known in the art. Fragments containing antigenic sequences may be selected on the basis of generally accepted criteria of potential antigenicity and/or exposure. Such criteria include the hydrophilicity and relative antigenic index, as determined by surface exposure analysis of proteins. The determination of appropriate criteria is well-known to one of skill in the art, and has been described, for example, by Hopp et al., Proc Natl Acad Sci USA 1981; 78: 3824-8; Kyte et al., J Mol Biol 1982; 157: 105-32; Emini, J Virol 1985; 55: 836-9; Jameson et al., CA BIOS 1988; 4: 181-6; and Karplus et al., Naturwissenschaften 1985; 72: 212-3. Amino acids domains predicted by these criteria to be surface exposed may be selected preferentially over domains predicted to be more hydrophobic. Given the hydrophobic central region of a signal peptide it may be necessary to include hydrophobic residues in the antigenic fragment.

Signal peptides or portions of a signal peptide may be chemically synthesized by methods known in the art from individual amino acids. Suitable methods for synthesizing protein fragments are described by Stuart and Young in “Solid Phase Peptide Synthesis,” Second Edition, Pierce Chemical Company (1984).

If a signal peptide or portion of a signal peptide defines an epitope, but is too short to be antigenic, it may be conjugated to a carrier molecule in order to produce antibodies. Some suitable carrier molecules include keyhole limpet hemocyanin, Ig sequences, TrpE, and human or bovine serum albumen. Conjugation may be carried out by methods known in the art. One such method is to combine a cysteine residue of the fragments with a cysteine residue on the carrier molecule.

Polyclonal and monoclonal antibodies may be produced by methods known in the art. Monoclonal antibodies may be produced by hybridomas prepared using known procedures including the immunological method described by Kohler and Milstein, Nature 1975; 256: 495-7; and Campbell in “Monoclonal Antibody Technology, The Production and Characterization of Rodent and Human Hybridomas” in Burdon et al., Eds. Laboratory Techniques in Biochemistry and Molecular Biology, Volume 13, Elsevier Science Publishers, Amsterdam (1985); as well as by the recombinant DNA method described by Huse et al, Science 1989: 246: 1275-81.

Other embodiments include functional equivalents of antibodies, and include, for example, chimerized, humanized, and single chain antibodies as well as fragments thereof. Methods of producing functional equivalents are disclosed in PCT Application WO 93/21319; European Patent Application No. 239,400; PCT Application WO 89/09622; European Patent Application 388,745; and European Patent Application EP 332,424.

Functional equivalents include polypeptides with amino acid sequences substantially the same as the amino acid sequence of the variable or hypervariable regions of the antibodies of the invention. “Substantially the same” amino acid sequence is defined herein as a sequence with at least 70%, preferably at least about 80%, and more preferably at least 90% homology to another amino acid sequence as determined by the FASTA search method in accordance with Pearson and Lipman, Proc Natl Acd Sci USA 1988; 85: 2444-8.

Chimerized antibodies may have constant regions derived substantially or exclusively from human antibody constant regions and variable regions derived substantially or exclusively from the sequence of the variable region from a mammal other than a human. Humanized antibodies may have constant regions and variable regions other than the complement determining regions (CDRs) derived substantially or exclusively from the corresponding human antibody regions and CDRs derived substantially or exclusively from a mammal other than a human.

Suitable mammals other than a human may include any mammal from which monoclonal antibodies may be made. Suitable examples of mammals other than a human may include, for example, a rabbit, rat, mouse, horse, goat, or primate.

Single chain antibodies or Fv fragments are polypeptides that consist of the variable region of the heavy chain of an antibody linked to the variable region of the light chain with or without an interconnecting linker. Thus, the Fv comprises the entire antibody combining site. These chains may be produced in bacteria.

Functional equivalents may further include fragments of antibodies that have the same, or binding characteristics comparable to the whole antibody. Such fragments may contain one or both Fab fragments or the F(ab′)2 fragment. Antibody fragments may contain all six complement determining regions of the whole antibody, although fragments containing fewer than all of such regions, such as three, four, or five CDRs, are also functional.

The invention provides a method for detecting a disease by determining the presence of a signal peptide, which corresponds to a marker protein for the disease, in a subject's bodily fluid. The invention also provides methods for quantitating and diagnosing a disease by measuring the amount of a signal peptide in the subject's bodily fluid, for which the parent protein is a marker of the disease. In certain embodiments, the measured amount of a signal peptide may be compared with the normal amount (i.e. the amount of protein present in a subject indicative of the absence of a disease that is associated with the up-regulation of the protein that is indicated by the signal peptide). Normal amounts may be determined from population studies of subjects free from a defined disease or from a previously determined reference range in such subjects. In one embodiment, the normal amount may be determined when a subject is stabilized or is not suffering from or is suffering from a less severe condition. This allows the relative change of a marker in a subject to be determined. A subject may be matched for age and/or gender.

The level of a signal peptide may be detected and/or quantitated using an immunoassay. Such assays may be competitive or non-competitive immunoassays. Such assays, both homogeneous and heterogeneous, are well-known in the art, wherein the analyte to be detected is caused to bind with a specific binding partner, such as an antibody, which has been labeled with a detectable species, such as a latex or particle, a fluorescent moiety, a biotinylated moiety, an enzyme, an electrochemically active species, etc. Alternatively, the analyte could be labeled with any of the above species and competed with limiting amounts of specific antibody. The presence or amount of analyte present is then determined by detection of the presence or concentration of the label. Such assays may be carried out in the conventional way using a laboratory analyzer or with point of care or home testing device, such as the lateral flow immunoassay as described in EP291194.

In certain embodiments, an immunoassay is performed by contacting a sample from a subject to be tested with an appropriate antibody under conditions that facilitates immunospecific binding between the antibody and the signal peptide if present. In the context of the disclosed invention, “immunospecific” means that the antibody will bind specifically to signal peptide. Any suitable immunoassay can be used, including, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays.

For example, a signal peptide can be detected in bodily fluid by means of a two-step sandwich assay. In the first step, a capture reagent (e.g., an anti-marker antibody) is used to capture the signal peptide. The capture reagent can optionally be immobilized on a solid phase. In the second step, a directly or indirectly labeled detection reagent is used to detect the captured signal peptide. In one embodiment, the detection reagent is an antibody. In another embodiment, the detection reagent is a lectin. Any lectin that preferentially binds to the marker rather than to other proteins that share the antigenic determinant recognized by the antibody can be used for this purpose. In further embodiments, the chosen lectin binds to the marker with at least 2-fold, 5-fold or 10-fold greater affinity than to other proteins that share the antigenic determinant recognized by the antibody. A lectin that is suitable for detecting a given marker can readily be identified by methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174.

In other embodiments, a lateral flow immunoassay device may be used in the ‘sandwich’ format wherein the presence of sufficient marker in bodily fluid will cause the formation of a ‘sandwich’ interaction at the capture zone in the lateral flow assay. The capture zone as used herein may contain capture reagents such as antibody molecules, antigens, nucleic acids, lectins, and enzymes suitable for capturing a signal peptide and other markers described herein. The device may also incorporate one or more luminescent labels suitable for capture in the capture zone, the extent of capture being determined by the presence of analyte. Suitable labels include fluorescent labels immobilized in polystrene microspheres. Microspheres may be coated with immunoglobulins to allow capture in the capture zone.

Other assays that may be used in the methods of the invention include, but are not limited to, flow-through devices. In a flow-through assay, one reagent (usually an antibody) is immobilized to a defined area on a membrane surface. This membrane is then overlaid on an absorbent layer that acts as a reservoir to pump sample volume through the device. Following immobilization, the remainder of the protein-binding sites on the membrane are blocked to minimize non-specific interactions. When the assay is used, bodily fluid containing a marker specific to the antibody is added to the membrane and filters through the matrix, allowing the marker to bind to the immobilized antibody. In this step, the marker or protein is captured with a capture reagent such as an antibody. In a second step, the protein is detected with a detection reagent, such as a secondary antibody. In embodiments wherein the first reactant is an antibody, a tagged secondary antibody (an enzyme conjugate, an antibody coupled to a colored latex particle, or an antibody incorporated into a colored colloid) may be added or released that reacts with captured marker to complete the sandwich in the detection step. Alternatively, the secondary antibody can be mixed with the sample and added in a single step. If a marker is present, a colored spot develops on the surface of the membrane.

By virtue of the hydrophobicity of a portion of the signal peptide, it is possible to bind that portion to a hydrophobic surface (for example, a polystyrene or blotting membrane). The more hydrophilic portion of the signal peptide can then be detected by specific antibodies (for example, when peptide is directly immobilized onto polystyrene ELISA plates or when liquids containing the peptide have been drawn through specially constructed ELISA plates with blotting membrane constituting the floor of the wells of the plates).

In further embodiments, a second marker or antibody may be used in conjunction with antibody against a signal peptide to detect, quantitate and/or diagnosis a disease. In certain embodiments, where the disease is related to heart disease, natriuretic peptides including ANP, BNP, and CNP may be used as a second marker. Antibodies binding to ANP, BNP, and CNP can be obtained commercially. Examples of commercially available antibodies binding to ANP are mouse anti-human ANP monoclonal antibody (Biodesign International), rabbit anti-human ANP monoclonal antibody (Biodesign International), mouse anti-human ANP monoclonal antibody (Chemicon), rabbit anti-human ANP amino acids 95-103 antibody (Immundiagnostik), rabbit anti-human ANP amino acids 99-126 antibody (Immundiagnostik), sheep anti-human ANP amino acids 99-126 antibody (Immundiagnostik), mouse anti-human ANP amino acids 99-126 monoclonal antibody (Immundiagnostik) and rabbit anti-human a-ANP polyclonal antibody (United States Biological). Examples of commercially available antibodies binding to BNP are rabbit anti-human BNP polyclonal antibody (Biodesign International), rabbit anti-BNP amino acids 1-20 polyclonal antibody (Biodesign International), anti-human BNP monoclonal antibody (Immundiagnostik), and rabbit anti-human BNP amino acids 1-10 polyclonal antibody (Immundiagnostik). Examples of commercially available antibodies binding to CNP include rabbit anti-C-Type Natriuretic Peptide-22 (Phoenix Pharmaceuticals). In other embodiments, the second marker may be CRP. Examples of commercially available antibodies binding to CRP include goat anti-human CRP polyclonal antibody (Alpha Diagnostic International), anti-human CRP monoclonal antibody (Alpha Diagnostic International), and anti-human CRP monoclonal antibody (Hytest).

In another embodiment, a signal peptide may be used as a diagnostic marker to determine the stage or severity of an associated disease condition in a subject. Signal peptides may be detected and/or quantitated in combination with a second marker indicative of this disease in bodily fluid by use of an immunoassay. A diagnosis may be made based upon the results obtained from a healthy individual or individuals.

In an additional embodiment, a signal peptide may be used to identify subjects at risk for developing an associated disease. In this method, subjects with identified risk to develop said disease may be monitored for changes in a signal peptide amount quantitated from bodily fluid by an immunoassay.

In a further embodiment, the invention provides a method for monitoring the effect of therapy administered to a subject having a disease associated with an up- or down-regulated signal peptide. In this method, signal peptide levels may be detected and/or quantitated from bodily fluid by an immunoassay prior to the commencement of therapy to establish a base level for the patient. During the course of treatment, signal peptide levels will be monitored for deviations from this base level to indicate whether the therapy is effective.

4. Kits

The invention also provides a kit for detecting and/or quantitating a signal peptide and other markers in a subject's bodily fluid. Such a kit may be useful for detecting a disease with which the signal peptide is associated. Said kit may comprise one or more reagents for detecting and/or quantitating the level of signal peptide in bodily fluid. The one or more reagents may comprise an antibody that is immunospecific for a signal peptide. The kit may further comprise one or more reagents for measuring the level of a second marker indicative of the same disease.

A kit of the invention may additionally comprise one or more of the following: (1) instructions for using the kit for determining the level of a protein or a signal peptide; (2) a labeled binding partner to any antibody present in the kit; (3) a solid phase (such as a reagent strip) upon which any such antibody is immobilized; and (4) a label or insert indicating regulatory approval for diagnostic, prognostic, or therapeutic use or any combination thereof. If a labeled binding partner to the antibody is not provided, the antibody itself can be labeled with a detectable marker, e.g., a chemiluminescent, enzymatic, fluorescent, or radioactive moiety.

Exemplification

The invention, having been generally described, may be more readily understood by reference to the following examples, which are included merely for purposes of illustration of certain aspects and embodiments of the invention, and are not intended to limit the invention in any way.

Study Populations

8 normal controls and 24 heart failure patients were recruited. All normal controls had echocardiographically demonstrated ejection fractions above 50%. Heart failure patients had ejection fractions under 45%.

Assay for Signal Peptide of BNP (BNP-SP)

20 ml of peripheral venous blood was drawn into pre-chilled Na-EDTA (1.5 mg/ml blood) tubes containing 500 IU/ml aprotinin. After centrifugation at 3000 rpm at 4° C. for 15 min, plasma was separated and stored at −70° C. until assayed. Prior to assay of BNP signal peptide (BNP-SP), plasma was extracted on C₁₈ Sep-Pak (Waters) columns, and dried on a centrifugal evaporator.

An antibody to BNP-SP was raised in a rabbit, by conjugating the peptide sequence PQTAPSRALLLLL (amino acids 3-15 of the whole proBNP sequence including the signal sequence) (SEQ ID NO:8) via a C-terminal cysteine to keyhole limpet haemocyanin (with a heterobifunctional cross linker ε-maleimidocaproic acid N-hydroxysuccinimide ester), as described in Hughes et al, Clinical Science 1999; 96: 373-380. After monthly subcutaneous injections of the conjugate protein, IgG was prepared from the serum with Protein-A Sepharose. The standard used was the peptide sequence above, dissolved in 1 M acetic acid. Appropriate dilutions were made into ILMA (immunoluminometric assay) buffer consisting of 1.5 mmol/l NaH₂PO₄, 8 mmol/l Na₂HPO₄, 140 mmol/l NaCl, 1 mmol/l EDTA, 1 g/l bovine serum albumin 1, and 0.1 g/l azide.

ELISA plates were coated with 1 pmol per well of the peptide above, dissolved in phosphate buffered saline (PBS). After an overnight incubation, plates were washed with PBS and then blocked with 1% bovine serum albumin in PBS for 3 hours.

Plasma extracts and standards were reconstituted with ILMA buffer. Each specimen was reacted with 5 ng of the IgG specific for BNP-SP in duplicate. After incubation overnight at 4° C., the extracts and standards were then pipetted into the peptide coated wells in the ELISA plates. The pre-incubation of samples with antibody increased the sensitivity of the assay. After another 24 hours at 4° C., the amount of rabbit IgG that had bound to the wells was detected by washing the plates with PBS-Tween (0.1%) and incubating with biotinylated-anti-rabbit IgG (Sigma Chemical Company, Poole, UK), diluted 1: 10,000 in ILMA for 2 hours at room temperature. After another series of washes with PBS-Tween, the biotinylated anti-rabbit IgG that was bound was detected with Streptavidin labelled with methyl-acridinium ester (Ng et al, Clinical Science 2002; 102: 411-416). Chemiluminescence was measured following injections of hydrogen peroxide in nitric acid, and then sodium hydroxide with cetyl ammonium bromide, as described in Ng et al, Clinical Science 2002; 102: 411-416.

The lower limit of detection of BNP-SP was 29.8 fmol/ml with no cross reactivity with BNP or N-BNP. Intra- and interassay coefficients of variation were under 10%.

N-BNP assay

N-terminal proBNP (N-BNP) was assayed using a two-site non-competitive Immunoluminometric assay, as described in Omland et al, Circulation 2002; 106: 2913-2918.

Statistics

Data were analysed with an SPSS package (SPSS Inc, IL). Receiver operating characteristic curves (plotting sensitivity vs. 1-specificity) were constructed and areas under the curves determined. Areas near 1 indicate that the diagnostic test is very good at discriminating diseased patients from normal controls, whereas areas of around 0.5 indicate that the tests are of no use in discriminating diseased patients from normal controls.

Results

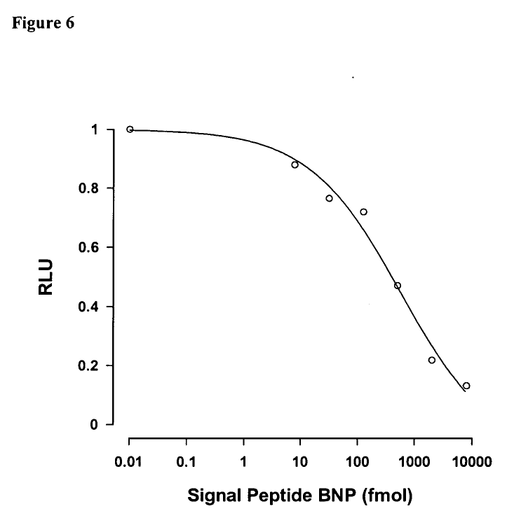

A typical standard curve is shown in FIG. 6, with half-displacement of binding at a 394 fmol of the signal peptide standard.

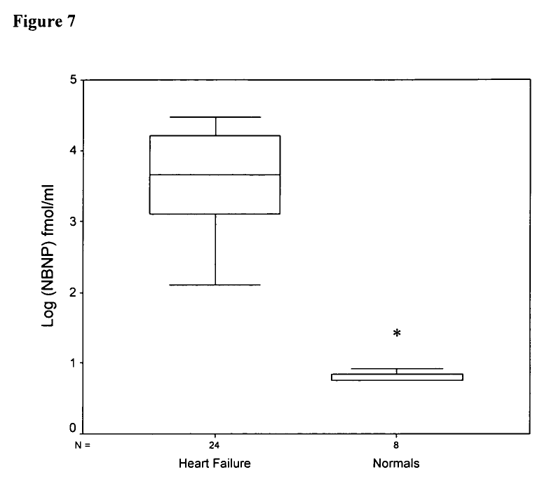

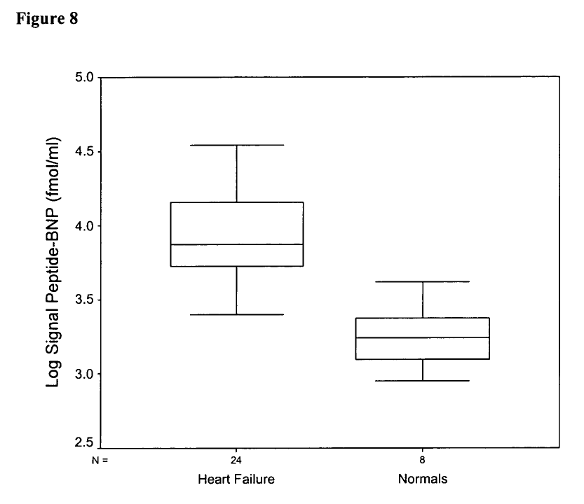

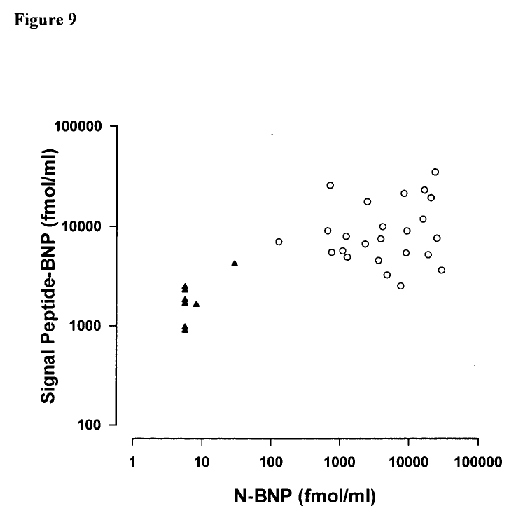

As expected, plasma levels of N-BNP were significantly elevated in the heart failure patients compared to normal control patients (P<0.0005 by Mann Whitney test, FIG. 7). The levels of BNP-SP were also very significantly elevated in the heart failure compared to control patients (P<0.0005 by Mann Whitney test, FIG. 8). The levels of BNP-SP were significantly correlated to those of N-BNP (Spearman rₛ=0.582, P<0.0005, FIG. 9).

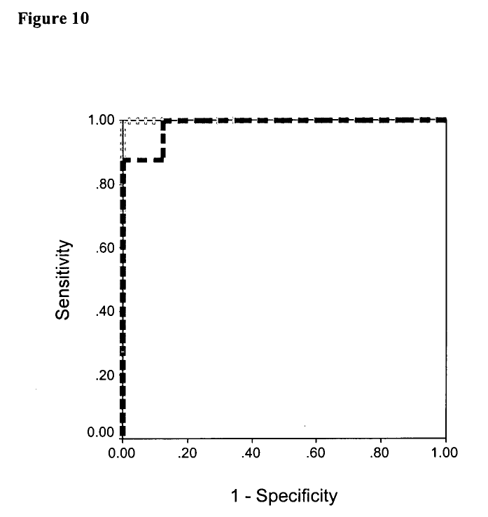

Areas under the receiver operating characteristic curves for diagnosis of heart failure were 1.000 (SEM 0) for N-BNP, and 0.984 (SEM 0.019) for BNP-SP. Both were significantly better than the diagonal (P<0.0005) which indicate that both tests are equally good at discriminating heart failure patients from normal subjects. The BNP-SP test has a specificity of 87.5% at 100% sensitivity (where all the heart failure cases are detected). The N-BNP test has 100% specificity at 100% sensitivity in this group of cases (FIG. 10).

Equivalents

The invention provides in part methods of diagnosing a disease in a subject by detecting and/or quantitating a signal peptide in a subject's bodily fluid. While specific embodiments of the subject invention have been discussed, the above specification is illustrative and not restrictive. Many variations of the invention will become apparent to those skilled in the art upon review of this specification. The appendant claims are not intended to claim all such embodiments and variations, and the full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

All publications and patents mentioned herein are hereby incorporated by reference in their entireties as if each individual publication or patent was specifically and individually indicated to be incorporated by reference. In case of conflict, the present application, including any definitions herein, will control.

Also incorporated by reference are the following: UK Patent Application No. 0325279.8; U.S. Pat. No. 5,955,311; U.S. Pat. No. 5,747,651; U.S. Pat. No. 4,816,567; U.S. Pat. No. 6,331,415; and WO 94/10202.

1. A method for diagnosing heart disease in a subject by detecting in the subject's bodily fluid the presence of a signal peptide selected from the group consisting of brain natriuretic peptide (BNP)-signal peptide, atrial natriuretic peptide (ANP)-signal peptide, C-type natriuretic peptide (CRP)-signal peptide, and C-reactive protein (CRP)-signal peptide.

2. The method of claim 1, wherein the detecting step is performed using an immunoassay.

3. The method of claim 1, wherein the detecting step is performed using a lateral flow immunoassay comprising the steps of capturing a signal peptide with a capture reagent and detecting the signal peptide with a detection reagent.

4. The method of claim 1, wherein the subject is human.

5. The method of claim 1, wherein the bodily fluid is plasma, whole blood, serum, interstitial fluid, urine, or saliva.

6. A method for diagnosing heart disease in a subject by quantitating in the subject's bodily fluid the amount of a signal peptide selected from the group consisting of brain natriuretic peptide (BNP)-signal peptide, atrial natriuretic peptide (ANP)-signal peptide, C-type natriuretic peptide (CRP)-signal peptide, and C-reactive protein (CRP)-signal peptide and comparing the amount to a normal value, wherein an amount higher than the normal value indicates that the subject has heart disease or is susceptible to developing heart disease.

7. The method of claim 6, wherein the quantitating step is performed using an immunoassay.

8. The method of claim 6, wherein the quantitating step is performed using a lateral flow immunoassay comprising the steps of capturing a signal peptide with a capture reagent and detecting the signal peptide with a detection reagent.

9. The method of claim 6, wherein the subject is human.

10. The method of claim 6, wherein the bodily fluid is plasma, whole blood, serum, interstitial fluid, urine, or saliva.

11. An antibody that binds brain natriuretic peptide (BNP)-signal peptide.

12. The antibody of claim 11, wherein the antibody is a polyclonal antibody.

13. A kit for detecting and/or quantitating a signal peptide in a subject's bodily fluid comprising at least one reagent that binds the signal peptide.

14. The kit of claim 13, wherein the reagent for detecting and/or quantitating a signal peptide is an antibody.

15. The kit of claim 13, further comprising a reagent that binds to a second marker of heart disease.
